(19) United States
(12) Patent Application Publication
(10) Pub. No.: US 2025/0017998 A1
(43) Pub. Date: Jan. 16, 2025

(54) SP-8356, EMODIN, AND IT603 ENHANCE BEIGE ADIPOCYTE ACTIVITY AND ALLEVIATE OBESITY AND DIABETES VIA UNCOUPLING PROTEIN 1 (UCP1)-INDEPENDENT MECHANISM

(71) Applicant: The Chinese University of Hong Kong, Hong Kong (CN)

(72) Inventors: Xiaoyan HUI, Hong Kong (CN); Jiaxing LIU, Hong Kong (CN); Qing LIU, Hong Kong (CN)

(21) Appl. No.: 18/763,336

(22) Filed: Jul. 3, 2024

Related U.S. Application Data
(60) Provisional application No. 63/511,911, filed on Jul. 5, 2023.

(51) Int. Cl. A61K 36/42; A61K 31/198; A61K 31/205; A61K 31/4375; A61K 31/593; A61K 33/04; A61K 33/06; A61K 33/24; A61K 33/30; A61K 36/27; A61K 36/54; A61P 3/06; A61P 3/10

(52) U.S. Cl. CPC A61K 36/42; A61K 31/198; A61K 31/205; A61K 31/4375; A61K 31/593; A61K 33/04; A61K 33/06; A61K 33/24; A61K 33/30; A61K 36/27; A61K 36/54; A61P 3/06; A61P 3/10

(57) ABSTRACT

The subject invention pertains to compositions and methods for the activation of beige adipocytes. The subject invention further pertains to methods of treating obesity, diabetes, and metabolic diseases. In certain embodiments, the subject composition comprises SP-8356 or a derivative thereof, emodin or a derivative thereof, IT603 or a derivative thereof, or any combination thereof.

### CROSS-REFERENCE TO RELATED APPLICATION

The present application claims the benefit of U.S. Provisional Application Ser. No. 63/511,911, filed Jul. 5, 2023, which is hereby incorporated by reference herein in its entirety, including any figures, tables, nucleic acid sequences, amino acid sequences, or drawings.

### REFERENCE TO SEQUENCE LISTING

The Sequence Listing for this application is labeled “CUHK-207CX-SeqList-03Jul24.xml” which was created on Jul. 3, 2024 and is 8,486 bytes. The entire content of the sequence listing is incorporated herein by reference in its entirety.

### BACKGROUND OF THE INVENTION

Obesity is a major public health issue. With the increasing social pressure, people are unconsciously consuming too many calories, and the proportion of obese people is increasing. Obesity is characterized by excessive accumulation of body fat. Current treatments for obesity or diabetes are limited, which include surgery and weight-loss pills. Surgery is an invasive treatment, which has many after-effects, including changes in the gastrointestinal digestive tract, severe nausea with persistent vomiting, heart palpitations, and sweating. Weight-loss pills usually have serious side effects, such as drowsiness, physical fatigue and even neurological disorders.

Beige adipocyte is a sub-type of adipocytes in the body that is capable of dissipating energy as heat. Therefore activation of beige adipocytes has been proposed as a new strategy to combat obesity and metabolic diseases. But safe and efficient approaches to activate beige adipocytes are yet to be identified.

Therefore, there remains a need for novel compositions and methods to achieve activation of beige adipocytes and therapeutic approaches for the treatment of obesity, diabetes, and metabolic diseases.

### BRIEF SUMMARY OF THE INVENTION

The subject invention pertains to compositions and methods for the activation of beige adipocytes. The subject invention further pertains to methods of treating obesity, diabetes, and metabolic diseases. In certain embodiments, the subject composition comprises SP-8356, emodin, IT603, or any combination thereof. In certain embodiments, SP-8356, emodin, IT603, or any combination thereof can significantly increase LETM1 domain containing 1 (LETMD1) expression, and energy expenditure in human beige adipocytes in a manner independent of Uncoupling protein 1 (UCP1). UCP1 is a mitochondria inner membrane protein, which has been regarded as the exclusive effector for energy dissipation in beige adipocytes. The invention targets beige adipocytes, which are a type of thermogenic adipocytes in the body that are able to consume excess energy. SP-8356, Emodin, and IT603 can induce mitochondrial protein LETMD1 expression, thus enhancing beige adipocyte activity and alleviating obesity, diabetes, and metabolic diseases in a UCP1-independent mechanism.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C. Generation and characterization of the LETMD1-tdTomato reporter hiPSC. (FIG. 1A) Schematic illustration of the knock-in design for the LETMD1-tdTomato reporter hiPSCs. (FIG. 1B) hiPSCs were differentiated into beige adipocytes and treated with β3 adrenergic receptor (β3-AR) agonist Mirabegron (0.1 μM) for 24 h and subjected to RNA-seq. The fold change of LETMD1 and its analogue LETM1 were plotted. (FIG. 1C) Representative images of tdTomato fluorescence intensity in LETMD1 reporter hiPSC-derived beige adipocytes treated with or without Mirabegron (0.1 μM) for 24 h.

FIGS. 2A-2F. SP-8356, emodin, and IT603 increase energy expenditure in human beige adipocytes in a UCP1-independent manner. WT or UCP1 knockout hiPSCs were differentiated to beige adipocytes followed by treatment with SP-8356 (1 μM), Emodin (1 μM), and IT603 (1 μM) for 24 h, respectively. DMSO was used as the vehicle control. The oxygen consumption rate (OCR) was examined by the Seahorse Mitochondrial Stress Test assay. (FIGS. 2A-2C) OCR in WT human beige adipocytes treated with SP-8356, Emodin, and IT603, respectively. (FIGS. 2D-2F) OCR in UCP1 knockout human beige adipocytes treated with SP-8356, Emodin, and IT603, respectively. *p<0.05, **p<0.01.

### BRIEF DESCRIPTION OF THE SEQUENCES

SEQ ID NO: 1: Nucleotide sequence encoding the FLAG cassette

SEQ ID NO: 2: Nucleotide sequence encoding IRES

SEQ ID NO: 3: Nucleotide sequence encoding tdTomato

SEQ ID NO: 4: Nucleotide sequence encoding T2A

SEQ ID NO: 5: Nucleotide sequence encoding hygromycin resistance gene

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the term “about” is used, these compositions contain the stated amount of the ingredient with a variation (error range) of 0-10% around the value (X±10%). In other contexts, the term “about” is used provides a variation (error range) of 0-10% around a given value (X±10%). As is apparent, this variation represents a range that is up to 10% above or below a given value, for example, X±1%, X±2%, X±3%, X±4%, X±5%, X 6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

“Treatment”, “treating”, “palliating” and “ameliorating” (and grammatical variants of these terms), as used herein, are used interchangeably. These terms refer to an approach for obtaining beneficial or desired results including but not limited to therapeutic benefit. A therapeutic benefit is achieved with the eradication or amelioration of one or more of the physiological symptoms associated with the underlying disease or symptom thereof such that an improvement is observed in the patient, notwithstanding that the patient may still be afflicted with the disease or symptom thereof.

As used herein, the term “obesity” refers to a chronic condition characterized as the increase in size and amount of fat cells in the body that can present a risk to health. Obesity can be measured using body mass index to screen for the chronic condition in an individual.

As used herein, the term “metabolic diseases” refers to a group of conditions which include cardiovascular diseases, fatty liver diseases, and/or diabetes. The conditions can include high blood pressure, high blood sugar, excess fat around the waist, and/or abnormal cholesterol or triglyceride levels.

As used herein, the terms “diabetes” or “diabetes mellitus” refers to a group of disorders characterized by excess sugar in the blood. Diabetes can include chronic diabetes, such as, for example type 1 diabetes or type 2 diabetes. Diabetes can also include prediabetes and gestational diabetes.

“Pharmaceutically acceptable salt” refers to a salt of a compound of the invention that is pharmaceutically acceptable and that possesses the desired pharmacological activity of the parent compound. In particular, such salts that are non-toxic may be inorganic or organic acid addition salts and base addition salts.

“Pharmaceutically acceptable vehicle” refers to a diluent, adjuvant, excipient or carrier with which a compound of the invention is administered. A “pharmaceutically acceptable vehicle” refers to a substance that is non-toxic, biologically tolerable, and otherwise biologically suitable for administration to a subject, such as an inert substance, added to a pharmacological composition or otherwise used to facilitate administration of an agent and that is compatible therewith. Examples of vehicles include but are not limited to calcium carbonate, calcium phosphate, various sugars and types of starch, cellulose derivatives, gelatin, vegetable oils, and polyethylene glycols.

The term “effective amount” or “therapeutically effective amount” refers to that amount of an compound described herein that is sufficient to affect the intended application, including but not limited to disease treatment. The therapeutically effective amount may vary depending on the intended application (in vitro or in vivo) or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., enhancing the activity of beige adipocytes. The specific dose will vary depending on the particular compounds chosen, the dosing regimen to be followed, whether it is administered in combination with other compounds, timing of administration, the tissue to which it is administered, and the physical delivery system in which it is carried.

In some embodiments of the invention, the method comprises administration of multiple doses of the compositions of the subject invention. The method may comprise administration of 1, 2, 3, 4, 5, 6, 7, 8, 9, 10, 15, 20, 25, 30, 35, 40, or more therapeutically effective doses of a composition of the subject invention as described herein. In some embodiments, doses are administered over the course of 1 day, 2 days, 3 days, 4 days, 5 days, 6 days, 7 days, 10 days, 14 days, 21 days, 30 days, or more than 30 days. Moreover, treatment of a subject with a therapeutically effective amount of the compositions of the invention can include a single treatment or can include a series of treatments. It will also be appreciated that the effective dosage of a composition used for treatment may increase or decrease over the course of a particular treatment. Changes in dosage may result and become apparent from the results of diagnostic assays or imaging techniques for detecting blood sugar concentration, blood pressure, heart disease, which are known in the art. In some embodiments of the invention, the method comprises administration of the composition several time per day, including but not limiting to 2 times per day, 3 times per day, and 4 times per day.

As used herein, the term “subject” refers to an animal, needing or desiring delivery of the benefits provided by a therapeutic composition. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; immune health; enhancement of the function of enamel, an organ, tissue, or system in the body. The subject can be of any age or stage of development, including infant, toddler, adolescent, teenager, adult, or senior. The terms “subject” and “patient” can be used interchangeably.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Compositions

In certain embodiments, the compositions and methods according to the subject invention utilize SP-8356 or a derivative thereof, emodin, or a derivative thereof, IT603, or a derivative thereof, or any combination thereof. In certain embodiments, SP-8356 is formula (I), emodin is formula (II), and IT603 is formula (III). In certain embodiments, a derivative of SP-8636 is formula (IV) or formula (V), a derivative of emodin is formula (VI) or formula (VII), and a derivative of IT603 is formula (VIII).

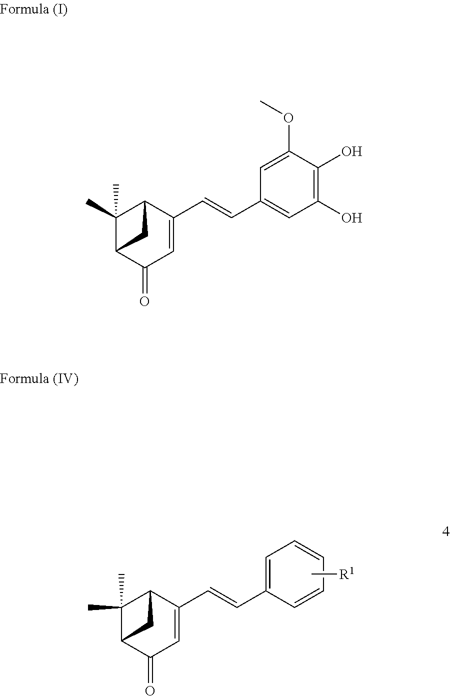

wherein
R¹ is H;
4′-F;
4′-OMe;
4′-Ph;
4′-pyrrol-1-yl;
3′,4′-diOMe;
3′,5′-diOMe; or
2′,5′-diOMe

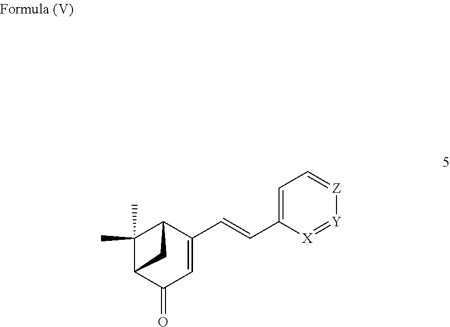

wherein
X is N, Y is C, and Z is C;
X is C, Y is N, and Z is C; or
X is C, Y is C, and Z is N.

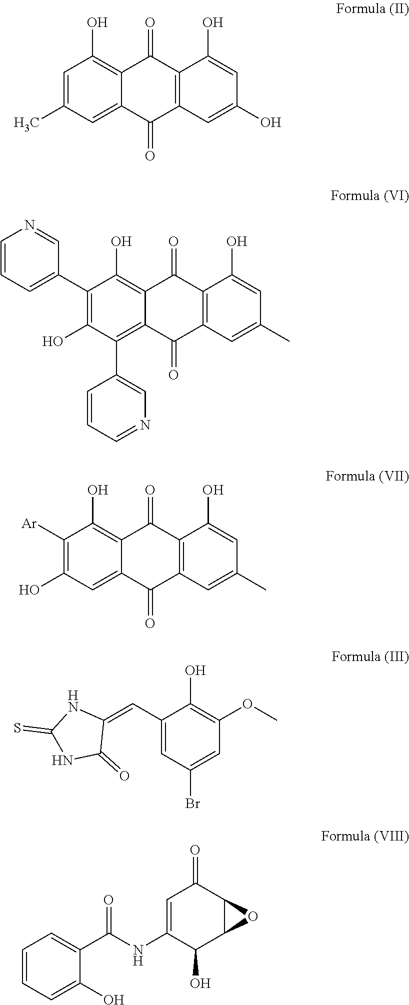

SP-8356 or a derivative thereof, emodin or a derivative thereof, or IT603 or a derivative thereof may be added to compositions at concentrations of about 0.0001 to about 50% by weight (wt %), preferably about 0.01 to about 10 wt %, and most preferably about 0.1% to about 1 wt %. In another embodiment, SP-8356 or a derivative thereof, emodin or a derivative thereof, IT603 or a derivative thereof, or any combination thereof can be in combination with an acceptable carrier and/or excipient, in that SP-8356 or a derivative thereof, emodin or a derivative thereof, IT603 or a derivative thereof, or any combination thereof may be presented at concentrations of about 0.0001 to about 50% (v/v), preferably, about 0.01 to about 10% (v/v), more preferably, about 0.1 to about 1% (v/v). In certain embodiments, SP-8356 or a derivative thereof, emodin or a derivative thereof, IT603 or a derivative thereof, or any combination thereof may be added to compositions at concentrations of about 0.01 μM to about 100 μM, about 0.1 μM to about 10 μM, about 0.5 μM to about 5 μM, or about 1 μM.

In some embodiments the subject invention provides pharmaceutically acceptable salts, solvates or hydrates thereof, of the compounds described herein.

Pharmaceutically acceptable salts can be a salt with an inorganic acid, such as hydrochloric acid, hydrobromic acid, perchloric acid, nitric acid, thiocyanic acid, sulfuric acid, and phosphoric acid; an organic acid, such as trifluoroacetic acid (TFA), formic acid, acetic acid, propionic acid, glycolic acid, lactic acid, pyruvic acid, oxalic acid, malonic acid, succinic acid, maleic acid, and fumaric acid; or a salt with a base, such as sodium hydroxide, ammonium hydroxide, potassium hydroxide, and organic bases such as mono-, di-, trialkyl and aryl amines, and substituted ethanolamines.

Further, pharmaceutically acceptable salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-di sulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

Hydrate refers to a compound of the present invention or a salt thereof, that further includes a stoichiometric or non-stoichiometric amount of water bound by non-covalent intermolecular forces.

Solvate means a solvate formed from the association of one or more solvent molecules to a compound of the present invention. The term “solvate includes hydrates (e.g., mono hydrate, dihydrate, trihydrate, tetrahydrate, and the like).

Certain embodiments provide amorphous forms of salts of the compounds disclosed herein. Such amorphous forms are advantageous for oral, pulmonary, buccal, intravaginal, or suppository delivery. In preferred embodiments, the subject compounds are administered orally.

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, intracerebroventricularly or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In one embodiment, the compositions of the subject invention can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the compositions of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

The subject invention also concerns kits comprising in one or more containers of at least 1, 2, 3, or more compounds of the subject invention. A kit of the invention can also comprise one or more compounds, biological molecules, or drugs. In one embodiment, a kit of the invention comprises a compound of the subject invention. In certain embodiments, the subject invention further pertains to a kit comprising the compounds of the subject invention, compositions comprising the subject compounds and, optionally, other compounds, including, for example, insulin effective in treating symptoms of diabetes, obesity, or metabolic diseases.

The invention further provides kits, including compounds of the subject invention and pharmaceutical formulations, packaged into suitable packaging material, optionally in combination with instructions for using the kit components, e.g., instructions for performing a method of the invention. In one embodiment, a kit includes an amount of compounds of the subject invention and instructions for administering the compounds of the subject invention to a subject in need of treatment on a label or packaging insert. In further embodiments, a kit includes an article of manufacture, for delivering the compounds of the subject invention into a subject locally, regionally or systemically, for example.

As used herein, the term “packaging material” refers to a physical structure housing the components of the kit. The packaging material can maintain the components sterilely and can be made of material commonly used for such purposes (e.g., paper, corrugated fiber, glass, plastic, foil, ampules, etc.). The label or packaging insert can include appropriate written instructions, for example, practicing a method of the invention, e.g., treating a diabetes, treating obesity, an assay for identifying a subject having diabetes, an assay for identifying a subject having obesity etc. Thus, in additional embodiments, a kit includes a label or packaging insert including instructions for practicing a method of the invention in solution, in vitro, in vivo, or ex vivo.

Instructions can therefore include instructions for practicing any of the methods of the invention described herein. For example, pharmaceutical compositions can be included in a container, pack, or dispenser together with instructions for administration to a subject to treat diabetes, obesity, and/or metabolic diseases. Instructions may additionally include appropriate administration route, dosage information, indications of a satisfactory clinical endpoint or any adverse symptoms that may occur, storage information, expiration date, or any information required by regulatory agencies such as the Food and Drug Administration or European Medicines Agency for use in a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD-or DVD-ROM/RAM, magnetic tape, electrical storage media such as RAM and ROM and hybrids of these such as magnetic/optical storage media.

Kits can additionally include a buffering agent, a preservative, or an agent for stabilizing the compounds of the subject invention. The kit can also include control components for assaying for the presence of blood sugar, e.g., a control sample or a standard. Each component of the kit can be enclosed within an individual container or in a mixture and all of the various containers can be within single or multiple packages.

### Methods of Using the Subject Compositions

In certain embodiments, SP-8356 or a derivative thereof, emodin or a derivative thereof, IT603 or a derivative thereof, or any combination thereof can be administered to a subject. Any means of administration that can permit SP-8356 or a derivative thereof, emodin or a derivative thereof, IT603 or a derivative thereof, or any combination thereof to contact beige adipocyte cells in a subject, including, for example, orally, intravenously, intraperitoneally, intramuscularly, intrathecally, or subcutaneously are envisioned in the subject methods.

In certain embodiments, SP-8356 or a derivative thereof, emodin or a derivative thereof, IT603 or a derivative thereof, or any combination thereof can increase or enhance the activity of beige adipocyte cells. SP-8356 or a derivative thereof, emodin or a derivative thereof, IT603 or a derivative thereof, or any combination thereof can increase or enhance the activity of beige adipocyte cells by modulating the expression of LETM1 domain containing (LETMD1), preferably increasing the expression of LETMD1. In certain embodiments, administration of compositions of the subject invention can result in an increase in the expression of LETMD1 by about 5% to about 75%, about 10% to about 60%, about 20% to about 50%, about 30% to about 50%, about 40% to about 50%, or about 50% relative to a beige adipocyte cell that has not been treated with the subject compounds.

UCP1 is a mitochondria inner membrane protein, which has been regarded as the exclusive effector for energy dissipation in beige adipocytes. In certain embodiments, the subject methods increase or enhance energy expenditure in human beige adipocytes in a manner independent of Uncoupling protein 1 (UCP1). In certain embodiments, the subject compositions can enhance energy dissipation in UCP1-independent mechanism, and thus, the compositions can be applied to subjects that have UCP1-positive adipocytes. In certain embodiments, the compositions can be applied to subjects that have UCP1-negative adipocytes. In certain embodiments, the compositions can be applied to subjects that have UCP1-positive and UCP1-negative adipocytes.

In certain embodiments, the energy expenditure of a beige adipocyte treated with the subject composition can be increased by about 5% to about 75%, about 10% to about 60%, about 20% to about 50%, about 30% to about 50%, about 40% to about 50%, or about 50% relative to a beige adipocyte cell that has not been treated with the subject compounds. In certain embodiments, the energy expenditure can be measured by the oxygen consumption rate (OCR).

In certain embodiments, the subject compounds can be administered to a subject before the subject is diagnosed with diabetes (e.g., prediabetes), obesity, or metabolic diseases or to treat a subject diagnosed with diabetes, obesity, or metabolic diseases. In certain embodiments, the compounds of the subject invention can be administered in combination with insulin. In certain embodiments, the dosage of insulin can be about 80 mg/dL to about 130 mg/dL daily. In certain embodiments, the compounds of the subject invention or compositions thereof can be administered concurrently with insulin, before an administration of insulin, or after an administration of insulin to the subject. In certain embodiments, the administration of insulin occurs less than or about 15 minutes before or after the administration of the composition. In preferred embodiments, the compounds of the subject invention or compositions thereof can be administered concurrently with insulin.

Therapeutic or prophylactic application of the subject compounds and compositions containing the compounds thereof, can be accomplished by any suitable therapeutic or prophylactic method and technique presently or prospectively known to those skilled in the art. The compounds can be administered by any suitable route known in the art including, for example, oral, intramuscular, intraspinal, intracranial, nasal, rectal, parenteral, subcutaneous, or intravascular (e.g., intravenous) routes of administration. In preferred embodiments, the compound or composition thereof can be administered orally. Administration of the compounds of the invention can be continuous or at distinct intervals as can be readily determined by a person skilled in the art.

In some embodiments, an amount of the compounds can be administered 1 time per day, for 1, 2, 3, 4, 5, 6, 7, or more days. Treatment can continue as needed, e.g., for several weeks, for several months, for several years. For example, in some embodiments, an initial loading dose in the range of about 0.01 μM to about 100 μM, about 0.1 μM to about 10 μM, about 0.5 μM to about 5 μM, or about 1 μM is administered every day for 28, 35, 42, 49, 56, 100, 150, 200, 250, 300, 356, 500, 1000, 1500, 2000, 3000 or more days.

### Materials and Methods

Differentiation of Human Induced Pluripotent Stem Cell (hiPSC) to Beige Adipocytes

A total of 20 days was required for hiPSC differentiation into beige adipocytes. We used BMP4 (10 ng/mL) and ActivinA (25 ng/mL) from day 0 to day 4 to induce mesodermal differentiation in the first stage. We then induce mesodermal progenitors to differentiate into adipocyte progenitors with insulin (10 g/mL), isobutylmethylxanthine (IBMX) (500 μmol/L), dexamethasone (1 μmol/L), and indomethacin (50 μmol/L) from day 4 to day 10. Eventually, mature beige adipocytes will be formed by insulin (1 μg/mL) induction on day 10 to 20.

### Seahorse Assay

HiPSCs were plated in the XF96 plates (Agilent Technologies, Inc., Santa Clara, CA) and differentiated into the beige adipocytes. The XF Cell Mito Stress Test Kit (Seahorse Bioscience, 103015-100) was used to perform the assay by following the manufacturer's instructions.

Construction of a LETMD1 Reporter hiPSC

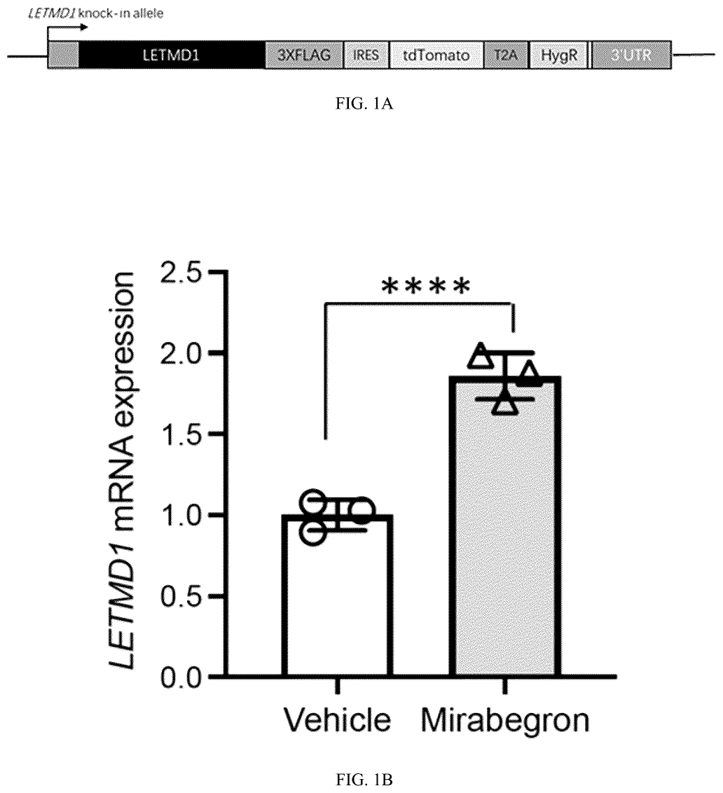

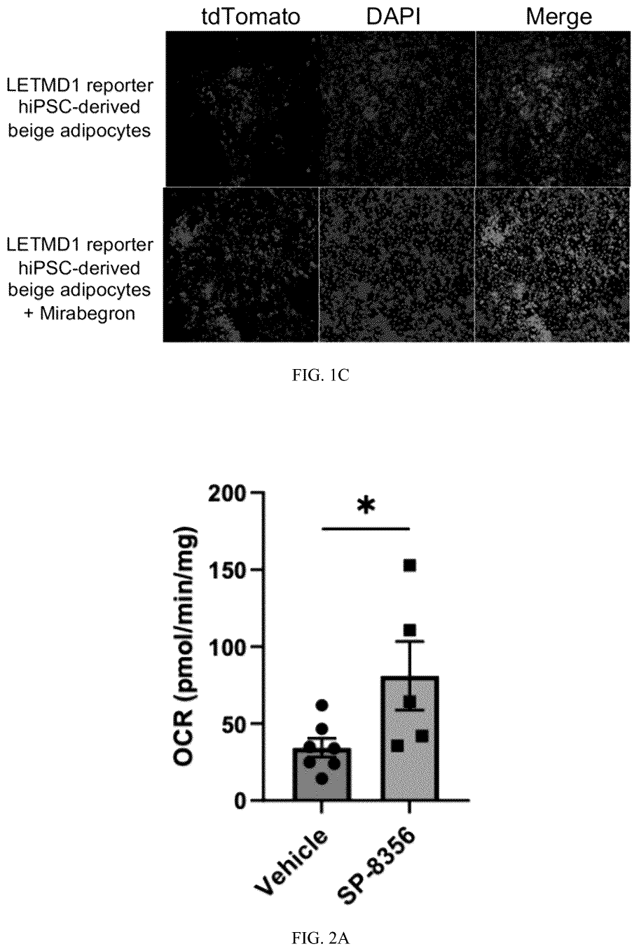

We generated a hiPSC LETMD1 reporter cell line using a CRISPR/Cas9-mediated strategy to insert a cassette encoding the 3x FLAG-IRES-tdTomato-2A-hygromycin resistance gene before the stop codon of LETMD1 gene (see FIG. 1A for the final gene locus).

In certain embodiments, FLAG is encoded according to SEQ ID NO: 1:
gactacaaggaccacgacggtgactacaaggaccacgacatcgactacaaggacgacgacgacaag.
In certain embodiments, IRES is encoded according to SEQ ID NO: 2:
ACGTTACTGGCCGAAGCCGCTTGGAATAAGGTCGGTGTGCGTTTGTCTATATGTTAT
TTTCCACCATATTGCCGTCTTTTGGCAATGTGAGGGCCCGGAAACCTGGCCCTGTCTT
CTTGACGAGCATTCCTAGGGGTCTTTCCCCTCTCGCCAAAGGAATGCAAGGTCTGTT
GAATGTCGTGAAGGAAGCAGTTCCTCTGGAAGCTTCTTGAAGACAAACAACGTCTGT
AGCGACCCTTTGCAGGCAGCGGAACCCCCCACCTGGCGACAGGTGCCTCTGCGGCC
AAAAGCCACGTGTATAAGATACACCTGCAAAGGCGGCACAACCCCAGTGCCACGTT
GTGAGTTGGATAGTTGTGGAAAGAGTCAAATGGCTCTCCTCAAGCGTATTCAACAAG
GGGCTGAAGGATGCCCAGAAGGTACCCCATTGTATGGGATCTGATCTGGGGCCTCG
GTGCACATGCTTTACATGTGTTTAGTCGAGGTTAAAAAACGTCTAGGCCCCCCGAAC
CACGGGACGTGGTTTTCCTTTGAAAAACACGATGATAATA
In certain embodiments, tdTomato is encoded according to SEQ ID NO: 3:
atggtgagcaagggcgaggaggtcatcaaagagttcatgcgcttcaaggtgcgcatggagggctccatgaacggccacgagttcgagat
cgagggcgagggcgagggccgcccctacgagggcacccagaccgccaagctgaaggtgaccaagggcggccccctgcccttcgcct
gggacatcctgtccccccagttcatgtacggctccaaggcgtacgtgaagcaccccgccgacatccccgattacaagaagctgtccttccc
cgagggcttcaagtgggagcgcgtgatgaacttcgaggacggcggtctggtgaccgtgacccaggactcctccctgcaggacggcacg
ctgatctacaaggtgaagatgcgcggcaccaacttcccccccgacggccccgtaatgcagaagaagaccatgggctgggaggcctccac
cgagcgcctgtacccccgcgacggcgtgctgaagggcgagatccaccaggccctgaagctgaaggacggcggccactacctggtgga
gttcaagaccatctacatggccaagaagcccgtgcaactgcccggctactactacgtggacaccaagctggacatcacctcccacaacga
ggactacaccatcgtggaacagtacgagcgctccgagggccgccaccacctgttcctggggcatggcaccggcagcaccggcagcgg
cagctccggcaccgcctcctccgaggacaacaacatggccgtcatcaaagagttcatgcgcttcaaggtgcgcatggagggctccatgaa
cggccacgagttcgagatcgagggcgagggcgagggccgcccctacgagggcacccagaccgccaagctgaaggtgaccaagggc
ggccccctgcccttcgcctgggacatcctgtccccccagttcatgtacggctccaaggcgtacgtgaagcaccccgccgacatccccgatt
acaagaagctgtccttccccgagggcttcaagtgggagcgcgtgatgaacttcgaggacggcggtctggtgaccgtgacccaggactcct
ccctgcaggacggcacgctgatctacaaggtgaagatgcgcggcaccaacttcccccccgacggccccgtaatgcagaagaagaccat
gggctgggaggcctccaccgagcgcctgtacccccgcgacggcgtgctgaagggcgagatccaccaggccctgaagctgaaggacgg
cggccactacctggtggagttcaagaccatctacatggccaagaagcccgtgcaactgcccggctactactacgtggacaccaagctgga
catcacctcccacaacgaggactacaccatcgtggaacagtacgagcgctccgagggccgccaccacctgttcctgtacggcatggacg
agctgtacaag.
In certain embodiments, T2A is encoded according to SEQ ID NO: 4:
GAGGGCAGAGGAAGTCTGCTAACATGCGGTGACGTCGAGGAGAATCCTGGCCCA
In certain embodiments, the hygromycin resistance gene is encoded according to SEQ ID
NO: 5:
atgaaaaagcctgaactcaccgcgacgtctgtcgagaagtttctgatcgaaaagttcgacagcgtctccgacctgatgcagctctcggagg
gcgaagaatctcgtgctttcagcttcgatgtaggagggcgtggatatgtcctgcgggtaaatagctgcgccgatggtttctacaaagatcgtt
atgtttatcggcactttgcatcggccgcgctcccgattccggaagtgcttgacattggggaattcagcgagagcctgacctattgcatctcccg
ccgtgcacagggtgtcacgttgcaagacctgcctgaaaccgaactgcccgctgttctgcagccggtcgcggaggccatggatgcgatcgc
tgcggccgatcttagccagacgagcgggttcggcccattcggaccgcaaggaatcggtcaatacactacatggcgtgatttcatatgcgcg
attgctgatccccatgtgtatcactggcaaactgtgatggacgacaccgtcagtgcgtccgtcgcgcaggctctcgatgagctgatgctttgg
gccgaggactgccccgaagtccggcacctcgtgcacgcggatttcggctccaacaatgtcctgacggacaatggccgcataacagcggt
cattgactggagcgaggcgatgttcggggattcccaatacgaggtcgccaacatcttcttctggaggccgtggttggcttgtatggagcagc
agacgcgctacttcgagcggaggcatccggagcttgcaggatcgccgcggctccgggcgtatatgctccgcattggtcttgaccaactcta
tcagagcttggttgacggcaatttcgatgatgcagcttgggcgcagggtcgatgcgacgcaatcgtccgatccggagccgggactgtcgg
gcgtacacaaatcgcccgcagaagcgcggccgtctggaccgatggctgtgtagaagtactcgccgatagtggaaaccgacgccccagc
actcgtccgagggcaaaggaatag.

### High-Throughput Drug Screening

LETMD1 reporter hiPSCs were plated in the 96-well plates and differentiated into beige adipocytes. The compounds from the compound library (Cat no. L3410, TargetMol, see worldwide website: targetmol.com/compound-library/Preclinical_Compound_Library) were added at a final concentration of 10 μM for 24 h. After that, the red fluorescent signals were quantified by the ThermoFisher (Waltham, MA) Varioskan Multimode Microplate Reader. Absorbance measurement for each compound was normalized to the vehicle control group.

### Animal Study

The mice were orally supplemented with the compound and the food intake, body weight were measured every three days. After 3 weeks of drug administration, indirect calorimetry was conducted by putting the mice into the metabolic cage (Promethion, Oxford, UK) for 3 days with access to food and water ad libitum at 21° C. and with a 12:12-hr light:dark cycle. The glucose tolerance of the mice was examined by glucose tolerance test.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Establishment of the LETMD1 Reporter Human Induced Pluripotent Stem Cells (HiPSCs)

To monitor the endogenous LETMD1 expression in human iPSCs with ease, we generated a fluorescent protein-based reporter human iPS cell line using a CRISPR/Cas9-mediated knock-in (KI) system (FIG. 1A). We have confirmed the validity of our reporter cell line by using a positive drug, Mirabegron. RNA sequencing result showed that the mRNA level of LETMD1 was significantly increased after treatment with the 03-adrenergic receptor (03-AR) agonist Mirabegron for 24 h after differentiation of hiPSCs into beige adipocytes (FIG. 1B). When we treated our reporter hiPSCs-derived beige adipocytes with Mirabegron, the density of tdTomato fluorescent protein increased significantly (FIG. 1C). This cell line enables us to monitor the endogenous expression of LETMD1 in a high-throughput manner using the red fluorescence intensity as a surrogate.

### Example 2—Drug Library Screening Results

A preclinical compound library, which contains 700 small compound candidates, was used to treat the LETMD1-tdTomato reporter beige adipocytes. 12 primary hits were obtained that significantly increased the expression level of LETMD1 in human beige adipocytes.

### Example 3—Functional Validation of the Screening Results

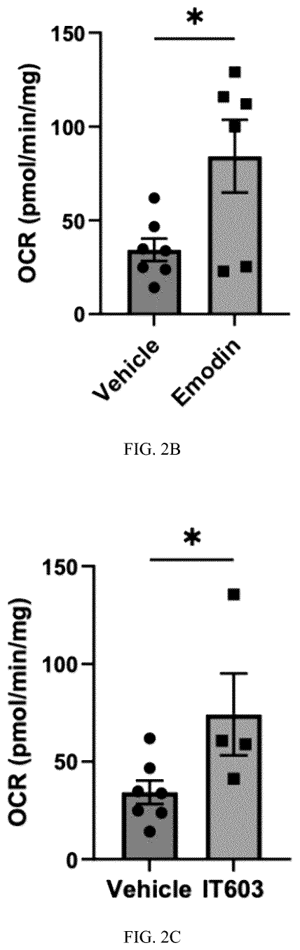

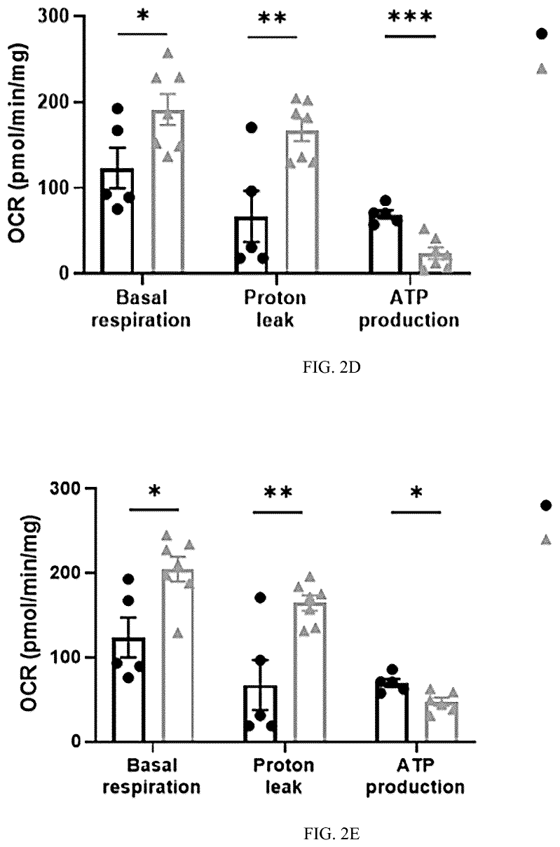

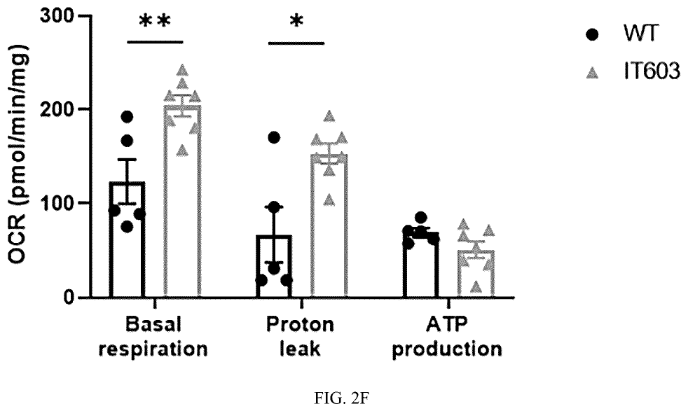

We then validated the effect of these 12 compounds on enhancing the metabolic rate of the beige adipocytes by measuring the oxygen consumption rate (OCR) of the adipocytes. Three compounds, SP-8356, emodin, and IT603, showed most significant capacity to elevate the OCR of the adipocytes (FIGS. 2A-2C). Furthermore, these three compounds enhanced the OCR in human beige adipocytes deficient of UCP1 (FIGS. 2D-2F). The results suggest that these three compounds hold the capacity to enhance the energy expenditure rate of beige adipocytes in a non-canonical, UCP1-independent manner. Therefore, they can be used as anti-obesity, anti-diabetes drugs. Therefore, the invention will provide a new method to achieve the activation of beige adipocytes and a therapeutic approach for the treatment of obesity, diabetes, and metabolic diseases.

### EXEMPLARY EMBODIMENTS

Embodiments of the subject invention include, but are not limited to, the following exemplified embodiments:

Embodiment 1. A method of activating beige adipocytes comprising administering an effective amount of a composition comprising SP-8356 or a derivative thereof, emodin or a derivative thereof, IT603 or a derivative thereof, or any combination thereof to a subject.

Embodiment 2. The method of embodiment 1, wherein the subject has diabetes, obesity, a metabolic disease, or any combination thereof.

Embodiment 3. The method of embodiment 1, wherein the composition increases an expression level of LETM1 domain containing (LETMD1).

Embodiment 4. The method of embodiment 3, wherein the expression level of LETMD1 is increased by at least about 10%, about 20%, about 30%, about 40%, about 50%, or about 60% in the subject.

Embodiment 5. The method of embodiment 1, wherein the concentration of SP-8356 or a derivative thereof is about 0.01 μM to about 10 μM.

Embodiment 6. The method of embodiment 1, wherein the concentration of emodin or a derivative thereof is about 0.01 μM to about 10 μM.

Embodiment 7. The method of embodiment 1, wherein the concentration of IT603 or a derivative thereof is about 0.01 μM to about 10 μM.

Embodiment 8. The method of embodiment 1, wherein the composition further comprises at least one carrier or excipient.

Embodiment 9. The method of embodiment 1, wherein the subject has Uncoupling protein 1 (UCP1)-positive adipocytes, UCP1-negative adipocytes, or a combination thereof.

Embodiment 10. The method of embodiment 1, wherein the derivative of SP-8356 is formula (IV) or formula (V):

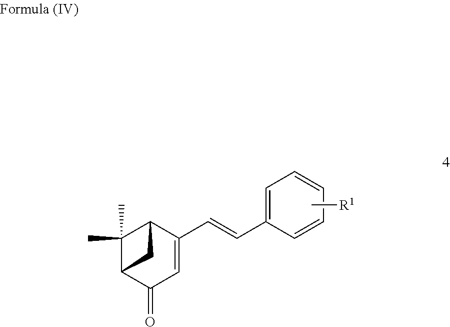

wherein
R¹ is H;
4′-F;
4′-OMe;
4′-Ph;
4′-pyrrol-1-yl;
3′,4′-diOMe;
3′,5′-diOMe; or
2′,5′-diOMe

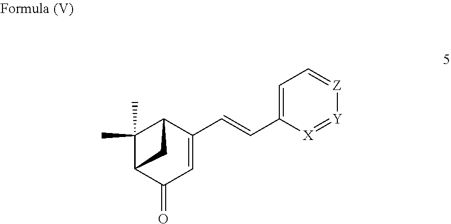

wherein
X is N, Y is C, and Z is C;
X is C, Y is N, and Z is C; or
X is C, Y is C, and Z is N.

Embodiment 11. The method of embodiment 1, wherein the derivative of emodin is formula (VI) or formula (VII):

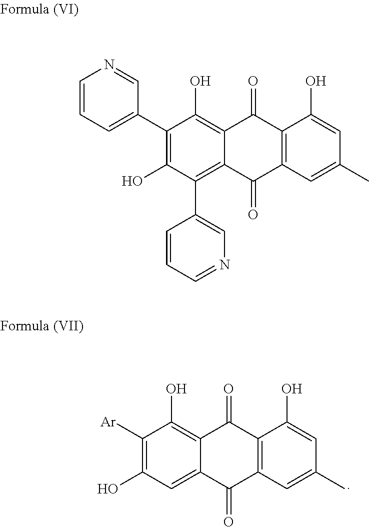

Embodiment 12. The method of embodiment 1, wherein the derivative of IT603 is formula (VIII):

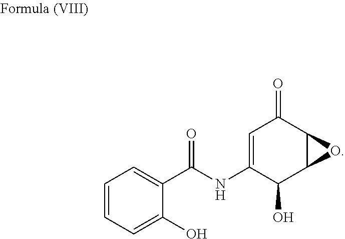 [SMILES: O=C(NC1=CC(=O)[C@H]2O[C@H]2[C@H]1O)c1ccccc1O]

Embodiment 13. A composition comprising SP-8356 or a derivative thereof, emodin or a derivative thereof, IT603 or a derivative thereof, or any combination thereof.

Embodiment 14. The composition of embodiment 13, wherein the composition comprises SP-8356 or a derivative thereof and emodin or a derivative thereof, emodin or a derivative thereof and IT603 or a derivative thereof, or SP-8356 or a derivative thereof and IT603 or a derivative thereof.

Embodiment 15. The composition of embodiment 13, wherein the composition comprises SP-8356 or a derivative thereof, emodin or a derivative thereof, and IT603 or a derivative thereof.

Embodiment 16. The composition of embodiment 13, wherein the composition further comprises at least one carrier or excipient.

Embodiment 17. The composition of embodiment 13, wherein the derivative of SP-8356 is Formula (IV) or Formula (V):

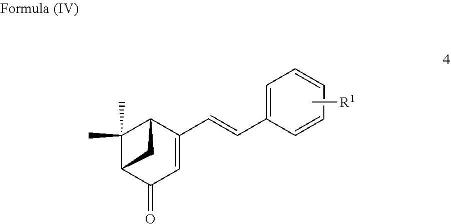

wherein
R¹ is H;
4′-F;
4′-OMe;
4′-Ph;
4′-pyrrol-1-yl;
3′,4′-diOMe;
3′,5′-diOMe; or
2′,5′-diOMe

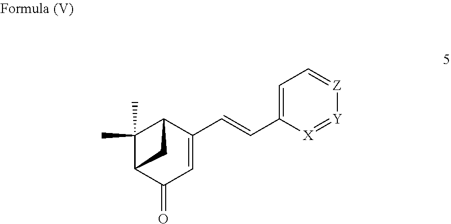

wherein
X is N, YisC, and Zis C;
X is C, Y is N, and Z is C; or
X is C, Y is C, and Z is N.

Embodiment 18. The composition of embodiment 13, wherein the derivative of emodin is Formula (VI) or Formula (VII):

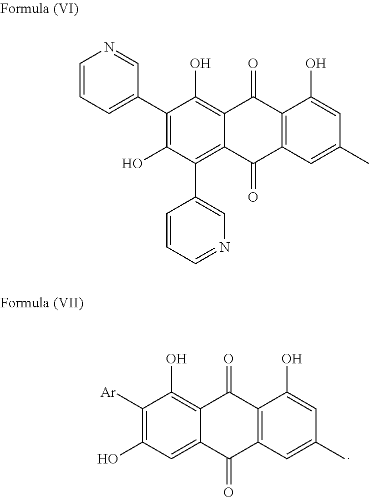

Embodiment 19. The composition of embodiment 13, wherein the derivative of IT603 is Formula (VIII)

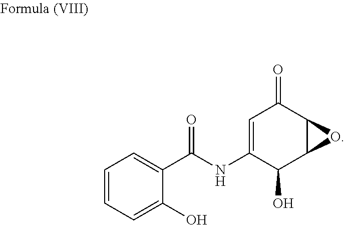 [SMILES: O=C(NC1=CC(=O)[C@H]2O[C@H]2[C@H]1O)c1ccccc1O]

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method of activating beige adipocytes comprising administering an effective amount of a composition comprising SP-8356 or a derivative thereof, emodin or a derivative thereof, IT603 or a derivative thereof, or any combination thereof to a subject.

2. The method of claim 1, wherein the subject has diabetes, obesity, a metabolic disease, or any combination thereof.

3. The method of claim 1, wherein the composition increases an expression level of LETM1 domain containing (LETMD1).

4. The method of claim 3, wherein the expression level of LETMD1 is increased by at least about 10%, about 20%, about 30%, about 40%, about 50%, or about 60% in the subject.

5. The method of claim 1, wherein the concentration of SP-8356 or a derivative thereof is about 0.01 μM to about 10 μM.

6. The method of claim 1, wherein the concentration of emodin or a derivative thereof is about 0.01 μM to about 10 μM.

7. The method of claim 1, wherein the concentration of IT603 or a derivative thereof is about 0.01 μM to about 10 μM.

8. The method of claim 1, wherein the composition further comprises at least one carrier or excipient.

9. The method of claim 1, wherein the subject has Uncoupling protein 1 (UCP1)-positive adipocytes, UCP1-negative adipocytes, or a combination thereof.

10. The method of claim 1, wherein the derivative of SP-8356 is formula (IV) or formula (V): 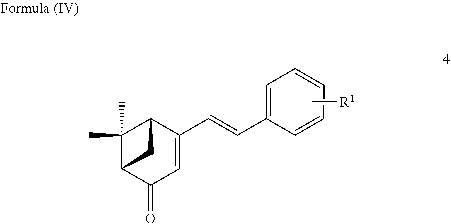
wherein
R¹ is H;
4′-F;
4′-OMe;
4′-Ph;
4′-pyrrol-1-yl;
3′,4′-diOMe;
3′,5′-diOMe; or
2′,5′-diOMe 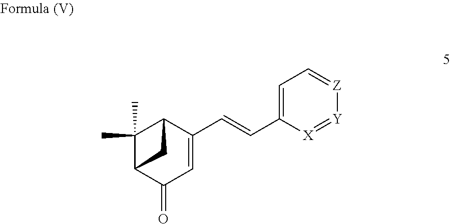
wherein
X is N, Y is C, and Z is C;
X is C, Y is N, and Z is C; or
X is C, Y is C, and Z is N.

11. The method of claim 1, wherein the derivative of emodin is formula (VI) or formula (VII):

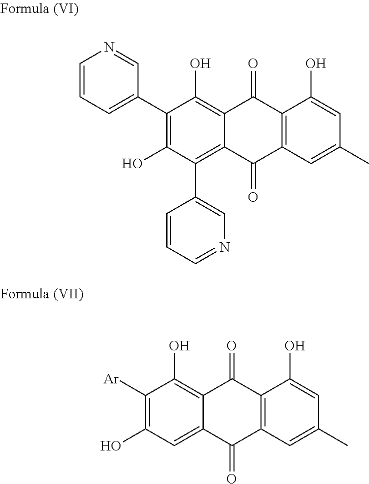

12. The method of claim 1, wherein the derivative of IT603 is formula (VIII):

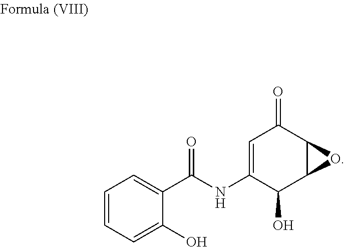 [SMILES: O=C(NC1=CC(=O)[C@H]2O[C@H]2[C@H]1O)c1ccccc1O]

13. A composition comprising SP-8356 or a derivative thereof, emodin or a derivative thereof, IT603 or a derivative thereof, or any combination thereof.

14. The composition of claim 13, wherein the composition comprises SP-8356 or a derivative thereof and emodin or a derivative thereof, emodin or a derivative thereof and IT603 or a derivative thereof, or SP-8356 or a derivative thereof and IT603 or a derivative thereof.

15. The composition of claim 13, wherein the composition comprises SP-8356 or a derivative thereof, emodin or a derivative thereof, and IT603 or a derivative thereof.

16. The composition of claim 13, wherein the composition further comprises at least one carrier or excipient.

17. The composition of claim 13, wherein the derivative of SP-8356 is Formula (IV) or Formula (V): 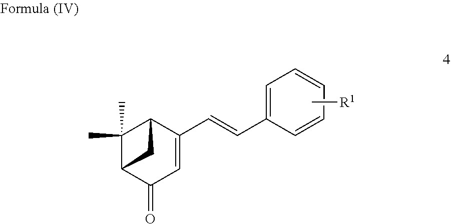
wherein
R¹ is H;
4′-F;
4′-OMe;
4′-Ph;
4′-pyrrol-1-yl;
3′,4′-diOMe;
3′,5′-diOMe; or
2′,5′-diOMe 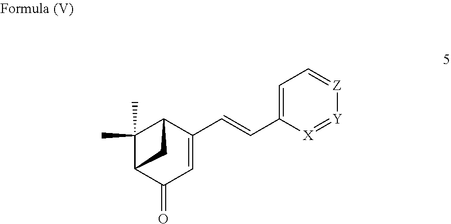
wherein
X is N, Y is C, and Z is C;
X is C, Y is N, and Z is C; or
X is C, Y is C, and Z is N.

18. The composition of claim 13, wherein the derivative of emodin is Formula (VI) or Formula (VII):

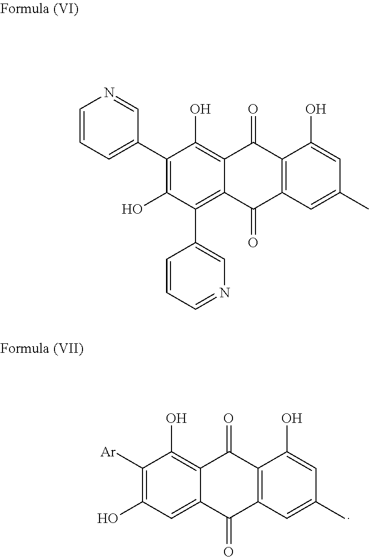

19. The composition of claim 13, wherein the derivative of IT603 is Formula (VIII) Formula (VIII): 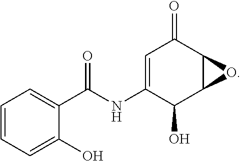 [SMILES: O=C(NC1=CC(=O)[C@H]2O[C@H]2[C@H]1O)c1ccccc1O]


SEQUENCE LISTING (5 sequences)

SEQ ID NO: 1 - DNA, 66 bp, synthetic construct
  source 1..66: mol_type = other DNA
gactacaaggaccacgacggtgactacaaggaccacgacatcgactacaaggacgacgacgacaag

SEQ ID NO: 2 - DNA, 551 bp, synthetic construct
  source 1..551: mol_type = other DNA
acgttactggccgaagccgcttggaataaggtcggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacgggacgtggttttcctttgaaaaacacgatgataata

SEQ ID NO: 3 - DNA, 1428 bp, synthetic construct
  source 1..1428: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaag

SEQ ID NO: 4 - DNA, 54 bp, synthetic construct
  source 1..54: mol_type = other DNA
gagggcagaggaagtctgctaacatgcggtgacgtcgaggagaatcctggccca

SEQ ID NO: 5 - DNA, 1026 bp, synthetic construct
  source 1..1026: mol_type = other DNA
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag